(19) United States
(12) Patent Application Publication
(10) Pub. No.: US 2001/0005717 A1
(43) Pub. Date: Jun. 28, 2001

(54) COMPLEXES FOR TRANSPORTING NUCLEIC ACID INTO EUKARYOTIC HIGHER-CELLS

(76) Inventors: ERNST WAGNER, LANGENZERSDORF (AT); MANFRED OGRIS, BIRMINGHAM (GB); RALF KIRCHEIS, VIENNA (AT); SYLVIA BRUNNER, VIENNA (AT)

Correspondence Address: STERNE KESSLER GOLDSTEIN & FOX, 1100 NEW YORK AVENUE NW, SUITE 600, WASHINGTON, DC, 20005-3934, US

(21) Appl. No.: 09/446,317

(22) PCT Filed: Jun. 18, 1998
(86) PCT No.: PCT/EP98/03679

§ 371 Date: None

(30) Foreign Application Priority Data
Jun. 20, 1997 (DE) 197 26 186.8

(51) Int. Cl. A61M 36/14; A61K 51/00; A61K 31/70; A61K 9/14

(52) U.S. Cl. 514/44; 435/458; 424/78.18; 424/486; 424/1.65

(57) ABSTRACT

Complexes of nucleic acid and polyethyleneimine (PEI), wherein PEI is modified with a hydrophilic polymer covalently coupled thereto, such as polyethyleneglycol, and processes for preparing them. A cellular ligand such as transferrin is optionally coupled to PEI. The complexes may be used for preparing pharmaceutical compositions for transferring therapeutically effective genes into mammalian cells.

The invention relates to the field of gene transfer.

It is known that the complexing of DNA with polyethyleneimine (PEI) can be used successfully for transporting genes into the cell (Boussif et al., 1995; Boussif et al., 1996; Abdallah et al., 1996). The gene transfer is carried out as a result of the complexes being bound to cells and taken up in an undirected manner. In order to make the binding specific, various ligands, e.g. transferrin (Tf) or antibodies are covalently coupled to PEI, in order to transport the genes into the cell through the mechanism of receptor-mediated endocytosis (Kircheis et al., 1997). However, even with this method, a certain proportion of the gene transfer achieved remains non-specific, which can be attributed to the uptake of the complexes into the cell independently of the ligand.

For efficient use of gene therapy in vivo other conditions besides specificity have to be satisfied. These include making the complexes as small as possible, for numerous applications. The need for the smallest possible complexes is caused inter alia by the physical conditions in the body, such as the small diameter of many blood vessels, for example; certain tissues can only be reached by small, non-aggregating complexes. If the complexes are to be taken up by receptor-mediated endocytosis, there is a size limit of not more than 200 nm, to allow uptake into the “coated pits” (Stryer, 1990).

Polycation/DNA-complexes have the advantage of low immunogenicity and lower risks over viral systems, but they are less efficient compared with viral gene transfer methods (Hodgson, 1995). This disadvantage can theoretically be cancelled out by using larger amounts of the DNA to be transferred. However, preliminary trials for the present invention have shown that increasing the concentration of DNA and polycation increases the tendency to aggregation during complexing.

Another limiting factor in gene transfer is the nonspecific immune response in the bloodstream of the body by so-called opsonisation, which is one of the first barriers which gene transfer particles have to overcome in vivo. Plasma proteins bind to any bacteria, viruses or other foreign bodies which have got in and trigger other defence mechanisms of the immune system (Roitt et al. 1991). The importance of protein binding to liposomes, as may be used for gene transfer, has been shown by Chonn et al., 1992. They were able to demonstrate a direct correlation between the amount of bound protein and the half-life of the liposomes in the bloodstream.

Another important component of the non-specific immune response is the activation of the complement system. Many cationic lipids and other polycations which are used for gene transfer exhibit a potent complement activation (Chonn, et al., 1991; Plank et al., 1996). Naturally occurring so-called dysopsonins may prevent attachment of these proteins (Absolom, 1986). Thus, for example, bacteria may counteract opsonisation by carrying highly hydrophilic sugar groups on their surface.

Various methods of preventing opsonisation of particles have already been developed. One of the methods most frequently used is the use of covalently coupled polyethyleneglycol (PEG) (Mori et al., 1991; Chonn et al., 1992; Woodle et al., 1994). This has been shown both to reduce protein binding and to prolong the half-life of the liposomes used in the bloodstream.

The amount of PEG used was mostly between 2 and 10% of PEG-coupled lipid in the liposome (m/m), the molecular weight of PEG was between 750 and 5000 D (Klibanov et al., 1990.; Blume et al, 1990; Mayhew et al, 1992; Papahadjopoulos et al, 1991; Senior et al, 1991; Mori et al, 1991; Yoshioka, 1991). Woodle et al., 1994, demonstrated the importance of the molecular weight for the steric stabilisation of particles. PEG derivatives ranging from 2,000 D to 5,000 D in size were found to be suitable; in the study by Torchilin et al., 1992, PEG derivatives with a molecular weight of 5,000 D were found to be suitable.

Klibanov et al 1991 described the stabilising effect of PEG 5000 D in liposomes which contain specific ligands (so-called immunoliposomes). However, it was also found that this PEG leads to rather poorer binding of the ligand to the receptor. In Torchilin et al 1992, however, it was shown that the longer half-life of the immunoliposomes as a result of PEG coating and hence a reduction in the non-specific uptake by the RES (reticuloendothelial system) more than compensates for the poorer ligand-receptor interaction.

Kirpotin et al., 1997, disclosed the use of bifunctional PEG'S, the preparation of which is described more fully by Zalipsky et al., 1997, for the subsequent coupling of ligands to PEG-liposomes.

Similar results to those obtained with PEG were also obtained for liposomes with gangliosides by Mori et al., 1991), and for polystyrene and gold particles with copolymers of polyoxyethylene and polyoxypropylene (Moghimi et al., 1993). In order to reduce the activation of the complement system, DNA/polylysine complexes were also modified with PEG (Plank et al., 1996). An increase in the specificity of so-called immunoliposomes has been demonstrated by Torchilin et al., 1992. Liposomes which contain both antibodies for a certain tissue and also PEG exhibit a clearly better specificity than liposomes without PEG.

Experiments by Torchilin et al., 1994, showed that amphiphilic vinylpolymers could significantly lengthen the half-life of liposomes in vivo. Torchilin and Papisov, 1994, showed that the mobility of the polymer chain would appear to be responsible for the protective effect of PEG and resultant longer half-life of liposomes.

The attempts made hitherto to reduce the interaction of DNA/polycation complexes with the complement system have been restricted to complexes containing polylysine (Plank et al., 1996). It was observed that the coupling of PEG to positively charged DNA/polylysine complexes can reduce the complement activation.

The problem of the present invention was to provide an alternative gene transfer system which is efficient and highly specific as well as being suitable for applications in vivo.

The solution to this problem consists of complexes of nucleic acid and polyethyleneimine, which are characterised in that the polyethyleneimine is modified with a hydrophilic polymer covalently coupled thereto.

The complexes according to the invention are hereinafter referred to as DNA/PEI/polymer complexes in the interests of simplicity.

The ratio of DNA to PEI is hereinafter given by stating the molar ratio of the nitrogen atoms in the PEI to the phosphate atoms in the DNA (N/P value); an N/P value of 6.0 corresponds to a mixture of 10 μg of DNA with 7.5 μg of PEI. In the case of free PEI, only about every sixth nitrogen atom is protonated under physiological conditions. Results with DNA/PEI complexes show that they are roughly electrically neutral at an N/P ratio of 2 to 3.

The N/P value of the complexes may fluctuate over a wide range; it may be within the range from about 0.5 to about 100. Preferably, the ratio is about 2 to about 20, most preferably the ratio is 3 to 10.

Specifically, the N/P value for the particular case, e.g. for the cell type which is to be transfected, may be determined by preliminary tests in which the ratio is increased under otherwise identical conditions in order to determine the optimum ratio in terms of the transfection efficiency and rule out any toxic effects on the cells.

The PEI contained in the complexes has a molecular weight of about 700 D to about 2,000,000 D. Larger PEI molecules yield optimum transfection efficiency after complexing with DNA even at lower N/P ratios, and result in very good transfection efficiency in general. Smaller molecules, of which a larger amount is needed for complexing, for the specified amount of DNA, have the advantage of lower toxicity, albeit with lower efficiency. Preliminary tests will show which PEI molecule should be used in each case.

PEI molecules with a molecular weight of between 2,000 and 800,000 are preferred within the scope of the present invention.

Examples of commercially obtainable PEI with different molecular weights which is suitable within the scope of the present invention are PEI 700 D, PEI 2000 D, PEI 25000 D, PEI 750000 D (Aldrich), PEI 50000 D (Sigma) and PEI 800000 D (Fluka). BASF also market PEI under the brand name Lupasol® in different molecular weights (Lupasol® FG: 800 D; Lupasol® G 20 anhydrous: 1300 D; Lupasol® WF: 25000 D; Lupasol® G 20: 1300 D; Lupasol® G 35: 2000 D; Lupasol® P: 750000 D; Lupasol® PS: 750000 D; Lupasol® SK: 2000000 D).

The hydrophilic polymer bound to PEI is preferably linear or branched only to a small extent, so that its mobility is largely maintained. (Without wishing to be tied to this theory, the beneficial effects of the polymer, besides its hydrophilicity, would appear to be attributable to its mobility.)

Examples of hydrophilic polymers coupled to PEI are selected from among polyethyleneglycols (PEG), polyvinylpyrollidones, polyacrylamides, polyvinylalcohols, or copolymers of these polymers.

The preferred hydrophilic polymer is PEG.

The molecular weight of the hydrophilic polymer is generally about 500 to about 20,000 D; molecules with a molecular weight of 1,000 to 10,000 D are preferably used.

The amount of polymer for coupling to PEI was determined using PEG in preliminary tests for the present invention by analysing the number of primary amines in the PEI molecule by ninhydrin assay (Sarin et al, 1981). It was established that about every tenth nitrogen atom occurs in the form of a primary amine. Therefore, a weight ratio of PEG-5000 D derivative to PEI of 9.2 was chosen as the starting point. This corresponds in order of magnitude to a molar ratio of PEG: primary amino groups/PEI molecules of 1:1.

The experiments carried out within the scope of the present invention as well as the accompanying tests showed that a molar ratio of polymer: primary amino groups/PEI in a range from 1:10 to 10:1 is suitable for the steric stabilisation of DNA/PEI complexes, depending on the particular application. The range is preferably from 1:5 to 5:1, most preferably from 1:3 to 3:1.

PEI is optionally modified with a cellular ligand in order to bring about the specific uptake of the complexes by binding to cell surface proteins, particularly receptors. Examples of ligands are given in WO 93/07283; transferrin or EGF is preferably used as the ligand.

The polymer molecule most suitable for a particular transfection according to type, molecular weight and amount can be determined in preliminary tests, as can the appropriateness of modifying PEI with a cellular ligand. In preliminary tests of this kind a given DNA/PEI complex is used as starting material and the nature and amount of the polymer is varied, then the stability of the complexes is compared under the transfection conditions selected. With respect to the need for or choice of a ligand, complexes which are identical apart from the presence or absence of a cellular ligand are compared with one another for their transfection efficiency.

The ligand is coupled to PEI by conventional methods, e.g. chemically, as described in WO 93/07283 for coupling virus, virus proteins or peptides with polyamine compounds.

In one embodiment of the invention, PEI is bound to the ligand via the hydrophilic polymer. This embodiment has the advantage that there are fewer restrictions with regard to the size of the polymer, as the accessibility of the ligand, which is found outside the polymer coating in this arrangement, and its binding to the receptor is not blocked by the polymer.

The nucleic acid contained in the complexes according to the invention is defined primarily by the biological effect to be achieved in the cell, or, when they are used in gene therapy, by the gene or gene section which is to be expressed, e.g. in order to substitute a defective gene, or by the target sequence of a gene which is to be inhibited. The nucleic acids to be transported into the cells may be DNAs or RNAs; there are no restrictions on the nucleotide sequence.

The complexes according to the invention have the advantage that they can be produced in a smaller size, and this effect is not affected by any PEI-coupled ligand.

The modification with PEG may also be carried out on larger complexes without affecting their functionality.

The invention further relates to a process for preparing the DNA/PEI polymer complexes.

DNA/PEI/polymer complexes may be prepared by various methods.

Preferably, DNA and PEI are first complexed by mixing the solutions and then, e.g. after a maturation period of about 20-40 minutes, the reaction with the polymer can take place (the “PEGylation” in the case of a reaction with PEG), as carried out in the Examples of the present invention. It has been established in the course of the present invention that complexing yields a significantly higher proportion of aggregated complex when there are high concentrations of the complex partners (cf Example 3c). It has been found that this frequently undesirable aggregation can be largely prevented by mixing the complexes from very dilute solutions. Reducing the salt concentration to below the physiological value reduces the effect of aggregate formation (Example 1). Using deionised water instead of physiological saline concentration can inhibit aggregation (Example 1). It has been found that physiological glucose concentrations have no effect on aggregate formation (cf FIG. 1). It was found that increasing the salt concentration to a level in the physiological range after the complexing does not negatively affect the stability of the complexes, while complexes without PEG rapidly formed aggregates (FIG. 2a). Moreover, it was found that the PEGylation of the complexes also leads to a reduced surface loading of the complexes (FIG. 14).

In an alternative preferred method the complexing is therefore carried out with low concentrations of the complexing partners, preferably about 5 to 50 μg of DNA/ml, particularly 10 to 40 μg of DNA/ml. The PEI concentration is matched to the DNA concentration, in accordance with the particular N/P value; it is e.g. 1.25 μg/ml of PEI 800000 D at an N/P value of 2 and a DNA concentration of 5 μg/ml; at a DNA concentration of 50 μg/ml corresponding to 12.5 μg/ml of PEI 800000 D. The complexing is also carried out at the lowest possible ion concentration, in order to prevent the formation of aggregates during the complexing or immediately afterwards. If desired, with a view to subsequent direct use of the complexes in vivo, the complexing is carried out in the presence of physiological sugar concentration (dextrose, glucose, saccharose).

The aggregation of the complexes is presumably inhibited by the formation of a thicker hydration shell which prevents the complexes from clumping together.

In an alternative method, complexes are obtained from dilute solutions using PEI which is already covalently coupled to the polymer, e.g. PEG (Example 2b). Here again, PEG has a stabilising effect, preventing the complexes from aggregating even after the addition of salt.

The covalent coupling of the polymer to PEI can be carried out by conventional methods, using polymer derivatives which are able to bind to the free amino groups of PEI. Various derivatives are commercially obtainable, e.g. the corresponding PEG derivatives (Shearwater Polymers, USA):

N-Hydroxysuccinimidyl active esters (Abuchowski et al, 1984; Klibanov et al, 1990 showed that the corresponding PEG derivatives could be used for the modification of liposomes); examples of commercially obtainable PEG derivatives of this type are methoxy-SS-PEG, MW 5000 D; methoxy-SSA-PEG, MW 5000 D); succinimidylsuccinate-propionic acid derivatives (methoxy-SPA-5000, MW 5000 D; methoxy-SPA-20000, MW 20000 D; methoxy-SSPA-PEG, MW 5000); oxycarbonylimidazole derivatives which react to form urethane (the binding of PEG derivatives of this type to proteins was demonstrated by Beauchamp et al, 1983, their use for the PEGylation of liposomes was shown by Allen et al, 1991; examples of commercial products are methoxy-PEG-CDI, MW 5000 D); glycidylethers (Pita et al, 1970; Elling et al, 1991); tresylates (the binding of PEG tresylates to proteins and liposomes was described by Nilsson et al, 1984; Yoshinaga et al, 1989; Delgado et al, 1990; Dust et al, 1990; Senior et al., 1991; Klibanov et al, 1991; examples of commercially obtainable PEG-tresylates are methoxy-PEG-Tres, MW 5000; methoxy-PEG-Tres, MW 200); aldehydes which are bound with sodium cyanoborohydride to amino groups (Wirth et al, 1991; commercial products are methoxy-PEG-ald, MW 5000; M-ALD-PEG-200: methoxy-PEG-ald, MW 2000).

If a cellular ligand is present in the complexes the following preparation method is used:

In one embodiment the PEI is coupled to the ligands as described in EP-A1 388 758 or by Kircheis et al., 1997, then the complexing is carried out with the other reactants, as described above.

In order to produce complexes in which the ligand is bound to PEI via the polymer, bifunctional polymers which have different reactive groups at both ends of the molecule are used. The polymers, e.g. PEG, which may be used for this are those used hitherto for the crosslinking of different macromolecules, e.g. for crosslinking cofactor and apoenzyme (Nakamura et al, 1986), controlling polymeric active substances (Zalipsky and Barany, 1990) or PEG-coating of surfaces and proteins (Harris et al, 1989). The bifunctional derivatives which may be used inter alia within the scope of the present invention are commercially obtainable; they contain amino groups, hydroxy groups or carboxylic groups at the ends of the molecule, e.g. such as the products obtainable from Shearwater Polymers. Other derivatives which may be used are NHS-maleinimide and NHS-vinylsulphone derivatives which react to their optimum at different pH values. Biotin-PEG-maleinimide or -NHS derivatives may also be used, whilst there may be a covalent coupling to the MAL or NHS group and the biotinylated end can react with molecules or particles containing streptavidin.

When bifunctional polymers are used there are a number of possible ways of forming DNA/PEI/ligand/polymer complexes: bifunctional polymer, e.g. PEG, may be coupled to PEI and a ligand with a suitable functional group may be coupled to the second, free functional group on the polymer, either before or after complexing with DNA, as desired. The PEG-PEI bond may be obtained via the primary amines of the PEI, although it is also possible to couple other reactive groups such as SH groups, which may act as reactants for PEG derivatives, to PEI beforehand. It is also possible to couple ligands to bifunctional PEG beforehand, whilst further bonding to PEI is possible before or after complexing with DNA. There are advantages in all these cases, particularly when using small ligands, which may be screened by the PEG during any subsequent PEGylation.

As a result of using bifunctional PEG derivatives the linear hydrophilic polymer molecule acts to some extent as a spacer between PEI and ligand.

For certain uses in vivo it is essential, with a view to achieving high gene transfer efficiency, for the complexes according to the invention to be present in a high concentration, usefully in a concentration of at least about 200 μg of DNA/ml. The complex concentration may be up to about 1 mg/ml, if there is a fairly high content of hydrophilic polymer.

The complexes according to the invention surprisingly have the advantage that they can be brought to the high concentration required from dilute solutions without any noticeable aggregate formation, which would affect the gene transfer efficiency. It has also been shown that the modification of the complexes with PEG leads to increased stability of the complexes in the blood of mice. This effect also helps gene transfer to take place in the subcutaneous tumour, e.g. after intravenous administration of the complexes.

In another aspect the invention relates to a composition for the transfection of higher eukaryotic cells, which contains DNA/PEI/PEG complexes in a concentration, based on DNA, of about 200 μg/ml to about 1 mg/ml.

In particular, the composition is present in the form of a pharmaceutical composition. In this embodiment the composition is used for transfection of mammalian cells in vivo; it contains as active ingredient a complex which contains a therapeutically active nucleic acid. Using the pharmaceutical composition according to the invention a high concentration of therapeutically active DNA can be achieved in the tissue by local administration. In systemic use the composition has the advantage that the complexes are not prone to either non-specific binding or degradation, thanks to the prevention of opsonisation.

By preventing or reducing non-specific binding and by introducing (cell-type-specific) cell-binding ligands into the complexes it is possible to target specific cells, organs or tissues (e.g. tumour tissue) and hence achieve targeted gene expression (e.g. in the tumour tissue) after systemic administration (Example 12).

Within the scope of the present invention it has been shown that, thanks to their longer circulation time in the blood, the complexes according to the invention stabilised by PEGylation are able to escape from the vascular system and into the surrounding tissue in areas of increased vascular permeability or damage to the blood vessels and accumulate there. Areas where such “passive targeting” occurs to a greater extent are tumours with a good blood supply and areas of inflammation.

The pharmaceutical composition may advantageously be used inter alia for the treatment of tumoral diseases, for intratumorally administering DNA containing a sequence, particularly on a plasmid, coding for one or more cytokines, such as interleukin-2, IFN-α, IFN-γ, TNF-α, or a suicide gene which is used in conjunction with the substrate, such as the Herpes simplex thymidine kinase gene (with ganciclovir) or the linamarase gene (with linamarin), or a DNA coding for an apoptosis-inducing protein, such as p53 or apoptin, or for a toxin such as the diphtheria toxin, or for an enzyme with a cytotoxic effect.

Another application in which the advantages of the composition according to the invention are demonstrated is so-called genetic tumour vaccination. The complexes used contain DNA, coding for one or more tumour antigens or fragments thereof, optionally combined with DNA coding for one or more cytokines.

The pharmaceutical composition according to the invention preferably occurs as a lyophilisate, optionally with the addition of sugar such as saccharose or dextrose in an amount which produces a physiological concentration in the solution ready for use. The composition may also be in the form of a cryoconcentrate.

The composition according to the invention may also be deep-frozen (cryopreserved) or in the form of a chilled solution.

In another aspect the invention relates to a process for preparing a composition for the transfection of mammalian cells, wherein complexes of dilute solutions of the complexing partners are first prepared and then brought to a concentration of at least 200 μg/ml.

The complexes may be concentrated by conventional methods, e.g. by ultrafiltration or by ultracentrifugation.

The compositions according to the invention may optionally be in the form of a kit having separate containers which hold the individual components DNA on the one hand and polymer-modified PEI, to which a ligand may optionally be coupled, on the other hand.

### SUMMARY OF FIGURES

FIG. 1: Suppressing aggregate formation in DNA/PEI complexes by mixing under salt-free conditions

FIG. 2: Stabilisation of DNA/PEI complexes with polyethyleneglycol (PEG)

a) covalent coupling of PEG after complexing of DNA with PEI

b) covalent coupling of PEG to PEI before complexing with DNA

c) dependency of particle size on the concentration of DNA and PEI in complex formation

FIG. 3: The covalent bonding of PEG is crucial to the stabilisation of the complexes

FIG. 4: Concentration of PEG-stabilised DNA/PEI complexes

FIG. 5: Interaction of DNA/PEI complexes with human plasma (Immunoblot)

FIG. 6: Reducing the protein binding to DNA/PEI complexes by modification with PEG

A) staining with silver

B) checking filterability

FIG. 7: Effect of PEG modification on gene transfer in K562 cells

FIG. 8: Effect of PEG modification on gene transfer in murine neuroblastoma cells

FIG. 9: Reducing the non-specific uptake of complexes by P388 mouse macrophages by modifying the complexes with PEG

FIG. 10: Reducing the interaction with plasma proteins by modifying DNA/Tf-PEI complexes with PEG

FIG. 11: PEGylation of DNA/TfPEI complexes increases the stability of the complexes in the blood after use in vivo

FIG. 12: Determining the biodistribution of PEGylated DNA/TfPEI complexes after systemic administration by Southern Blot

A) intact plus partly degraded reporter gene plasmid

B) intact reporter gene plasmid

FIG. 13: Targeted gene expression in the tumour tissue after the systemic administration of PEGylated DNA/TfPEI complexes

FIG. 14: Measurement of the zeta potential: reduced surface loading of PEGylated DNA/TfPEI and DNA/PEI complexes

FIG. 15: Effect of PEG modification of small and large complexes on gene transfer in mammalian cells

FIG. 16: Effect of PEG modification on EGF-mediated gene transfer in mammalian cells

### EXAMPLE 1:

### Suppressing aggregate formation in DNA/PEI complexes by mixing under salt-free conditions

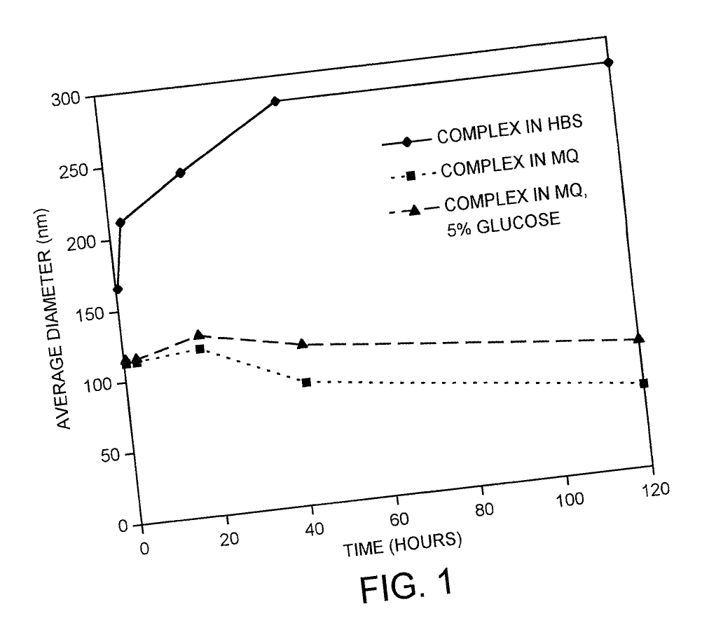

The complexes were formed by mixing equal volumes (250 μl) of dilute solutions of plasmid DNA, containing the sequence coding for the reporter gene luciferase (10 μg of the plasmid pCMVL, described in WO 93/07283) and 7.5 μg of PEI (N/P value: 6.0) or 9 μg of PEI (N/P value 7.2) by rapidly and repeatedly pipetting the solutions up and down, in order to mix the two components together as fast as possible. PEI with a molecular weight of 800000 Dalton was used (Fluka). The final concentration of DNA in the complex was 20 μg/ml. For complexes containing transferrin (Tf) conjugates with Tf covalently bound to PEI were used, the preparation of which was described by Kircheis et al., 1997. Two different conjugates were used: Tf2PEI (molar ratio of Tf/PEI 2/1) and Tf4PEI (molar ratio of Tf/PEI 4/1). The comparison of the complex mixture in HBS (150 mM NaCl, 20 mM HEPES, pH 7.3); in deionised water (MQ) on its own and in MQ with 5% glucose is shown in FIG. 1. The average particle size was measured at various times by quasielastic laser light scattering (Brookhaven BI-90). It was found that complexes in HBS aggregated after just a short time, whereas complexes which had been prepared in deionised water exhibited a stable size which was not substantially affected by a physiological glucose concentration.

### EXAMPLE 2:

### Stabilisation of DNA/PEI complexes with polyethyleneglycol (PEG)

a) Covalent coupling of PEG after complexing of the DNA with PEI

The DNA/PEI complexes with an N/P ratio of 6.0 were mixed as described in Example 1 and stored for 40 min at room temperature (RT) to complete the complexing. Then 69 μg of methoxy-succinimidyl-propionate-PEG (M-SPA-PEG, molecular weight of 5000 Dalton, Shearwater Polymers, Inc., USA, stock solution 10 mg/ml in DMSO) in 50 μl of MQ water were added. (A covalent bond was formed between M-SPA-PEG and the amino groups of the PEI.) The reaction took 20 min at RT; the weight ratio (w/w) of PEG to PEI was 9.2.

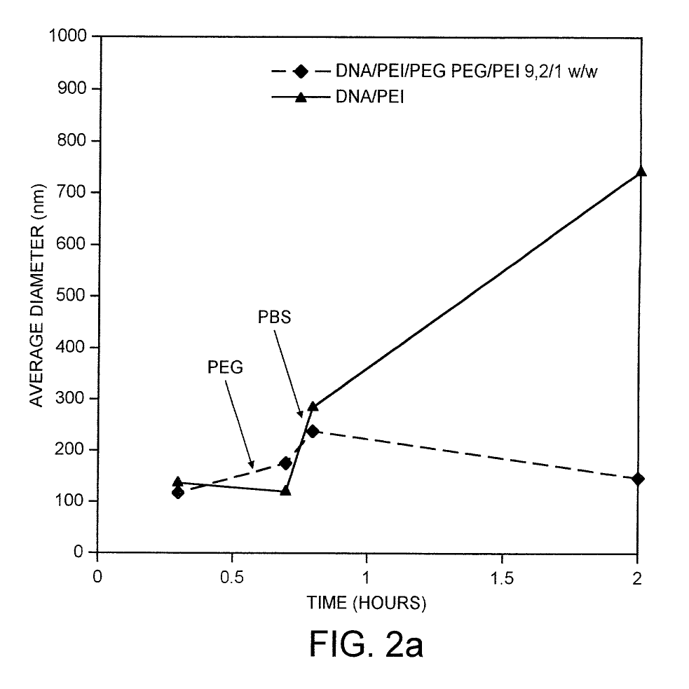

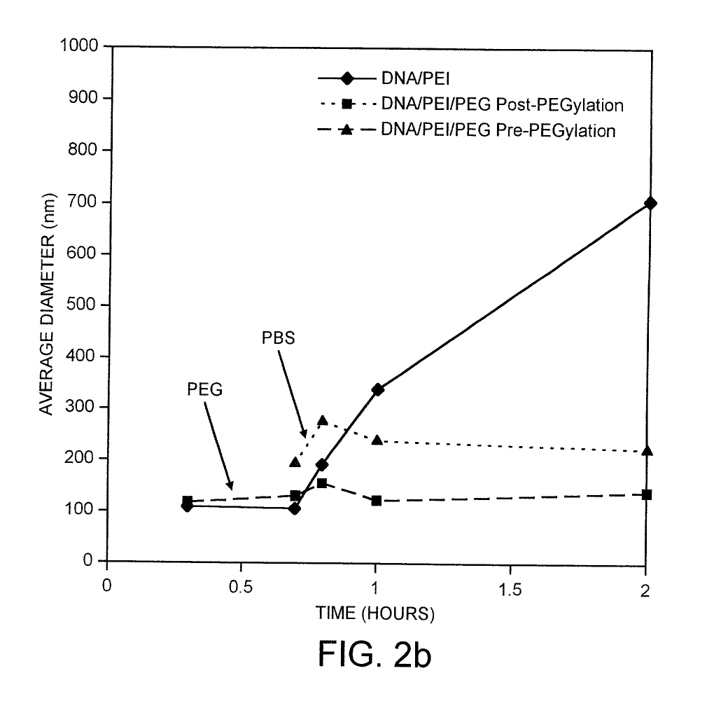

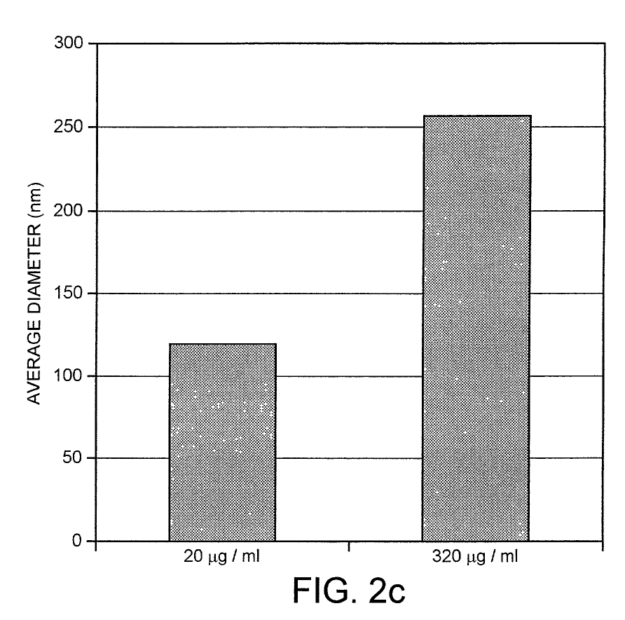

The complex size was measured at different times by quasielastic laser light scattering. In order to demonstrate the successful stabilisation of the complexes, a 250 μl aliquot of PBS (137 mM NaCl, 2.6 mM KCl, 6.6 mM Na₂HPO_{4,} 1.5 mM KH₂PO₄; pH 7.4) was added to the complex solution. This increase in the salt concentration caused the aggregation of sterically unstable complexes, whereas the PEG-modified complexes showed no change in size (FIG. 2a).

b) Covalent coupling of PEG to PEI before the complexing with DNA

The PEGylation of PEI before the complexing (“prePEGylation”) was carried out as follows: 7.5 μg of PEI were mixed with 6.9 μl of M-SPA-PEG 10 mg/ml in DMSO and the reaction was stopped after 20 min at RT by the addition of 0.2 μmol of glycine. (The free M-SPA-PEG still present reacts with the amino group of the glycine.) After another 20 min the solution was made up to 250 μl with MQ and complexed with 10 μg of DNA, as described in Example 2a. The rest of the procedure was as described in Example 2a.

The complexes used had an N/P value of 6.0, the ratio of PEG/PEI was 9.2 (w/w).

The subsequent PEGylation (“post-PEGylation”) of the complexes was carried out as described in Example 2a. The results show that sterically stable complexes can also be formed with previous PEGylation of PEI, but the average diameter of the particles is somewhat greater than with subsequent PEGylation (FIG. 2b).

c) Dependency of the particle size on the concentration and DNA and PEI during complexing

The complexes were mixed in MQ as described in Example 1, modified with PEG and the average particle diameter was measured by LLS. The DNA concentration during complexing was 20 or 320 μg/ml. The size was measured after PEGylation. It was clearly shown that more aggregates are formed by mixing in higher concentrations (FIG. 2c).

### EXAMPLE 3:

### The covalent binding of PEG is crucial to the stabilisation of the complexes

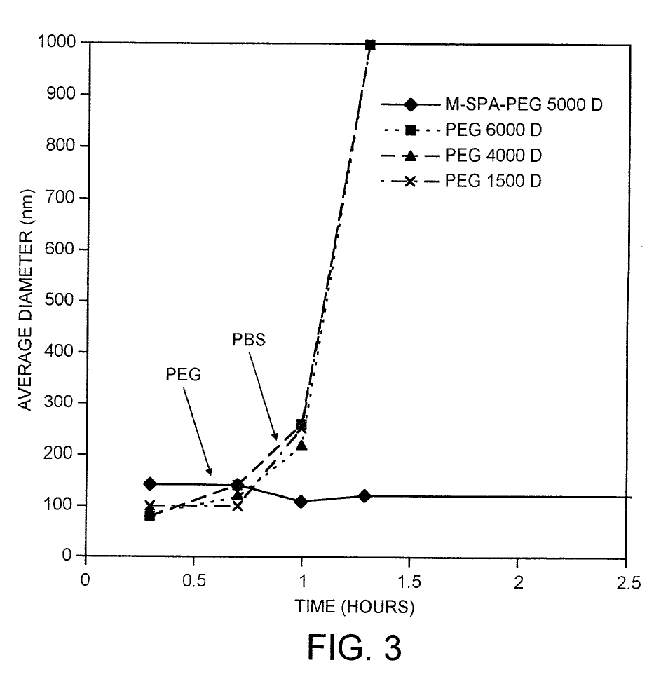

In this experiment a weight ratio of PEG to PEI of 9.2 was used. Methoxy-succinimidyl-propionate-PEG (M-SPA-PEG 5000) was used on the one hand, as in the previous Examples, whilst on the other hand PEG of a different molecular weight was used, with no reactive groups (6000 D: Merck, No. 807491; 4000 d: Loba Feinchemie, No. 81252; 1500 d: Merck, No. 807489) with average molecular weights of 6000, 4000 and 1500 Dalton. The size of the complex was measured at various times by quasielastic laser light scattering. After PEGylation a 250 μl aliquot of PBS was added to the complex solution. FIG. 3 shows that only covalent binding of PEG to the complex prevents the aggregation of the complexes after the addition of salt.

### EXAMPLE 4:

### Concentration of PEG-stabilised DNA/PEI complexes

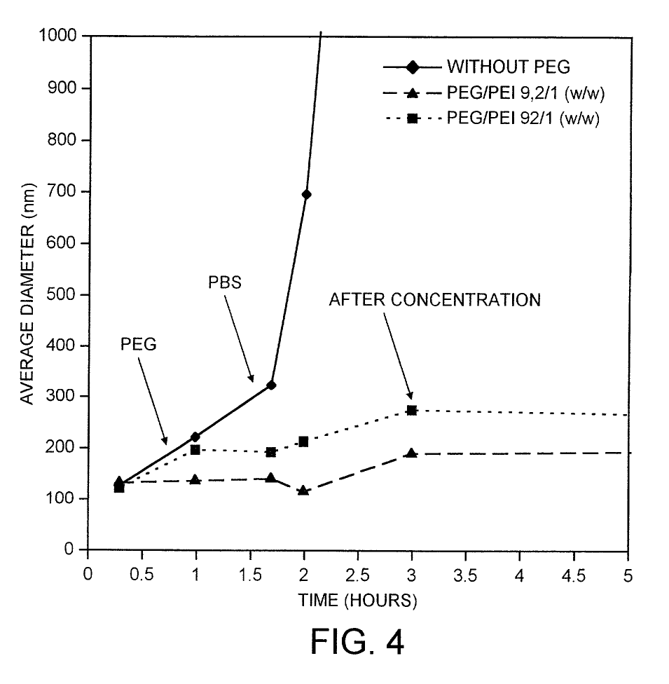

The complexes were mixed as described in Example 1, and stabilised with M-SPA-PEG as described in Example 2. After stabilisation and the addition of 250 μl of PBS, the complex solution (about 800 μl) was concentrated down to a volume of about 25 μl and hence a DNA concentration of about 400 μg/ml DNA using microconcentrators (Vivaspin 500, molecular exclusion volume 100,000 Dalton) at 12000 g. Then the concentration was re-adjusted to 20 μg/ml with MQ and the size was measured using quasielastic laser light scattering. FIG. 4 shows that without PEG modification after the concentration no reasonable particle sizes can be measured because of aggregation and/or absorption of the complexes onto the membrane, while the stabilised complexes also showed no aggregate formation after concentration.

### EXAMPLE 5:

### Interaction of DNA/PEI complexes with human plasma

This experiment served to determine the interaction of plasma proteins with the PEI complexes, whilst the proteins bound to the complexes were separated off together with them.

Human citrate plasma (Sigma) was used. In this experiment the complexes were mixed as follows: 12.8 μg of DNA in 20 μl MQ were mixed with 9.6 μg of PEI again in 20 μl MQ and modified as described in Example 2. Then the complexes were incubated with one aliquot of dilute plasma for 30 min at 37° C.

a) Identification of the plasma proteins binding to DNA/PEI complexes

In this experiment 40 μl of complex with a DNA concentration of 320 μg/ml were incubated with 140 μl of plasma diluted 1:70 for 30 min at 37° C. The complex/plasma solution was applied to microfiltration units with a filter pore size of 0.2 μm (Whatman, England, Anopore membrane). The membrane was saturated beforehand with a BSA solution (1 mg/ml) and washed three times with HBS (20 mM HEPES pH 7.3, 145 mM NaCl), to reduce non-specific protein binding. The solution applied was filtered at 12000 g and washed three times with HBS. The material left on the filter (complexes plus plasma proteins) was eluted with HBS+5% SDS (“eluate”) and, like the filtrate of the complex/plasma solution (“filtrate”), after the addition of one aliquot of five-fold concentrated non-reducing probe buffer (25% glycerol (w/v); 290 mM TRIS pH 6.8; 0.25% SDS (w/v); 0.1 mg/ml bromophenol blue), separated on an SDS-polyacrylamide gel with a polymer gradient of 2.5 to 12%.

For immunological identification of the proteins the gel was blotted in a “semi dry” blot apparatus (Bio Rad) on a nitrocellulose membrane, non-specific binding sites were saturated with a 1% solution of milk powder and incubated with the corresponding antibodies. The antibodies were diluted in TBST (150 mM NaCl; 10 mM TRIS pH 8.0; 0.1% TWEEN 20).

1st Antibody:

Goat anti-human complement C3 (fractionated antiserum, Sigma, Order no. C-7761, Lot Number 054H8842), dilution 1:3000.

Goat anti-human fibrinogen (fractionated antiserum, Sigma, Order no. F-2506, Lot Number 115H8828), dilution 1:3000. Goat anti-human fibronectin (fractionated antiserum, Sigma, Order no. F-1909, Lot Number 094H8868), dilution 1:3000.

2nd antibody:

Mouse anti-Goat IgG, HRP conjugated (polyclonal, Jackson Laboratories, Order no. 205-035-108, Lot Number 33740), dilution 1:25000

After incubation with the second antibody the nitrocellulose membrane was washed several times with TBST and then incubated in Luminol/Enhancer solution (Pharmacia, No. 1856135) and Stable Peroxide Solution (Pharmacia, No. 1856136) 1/1 (v/v) for 10 min at RT, washed several times with TBST and a film was exposed on the blot.

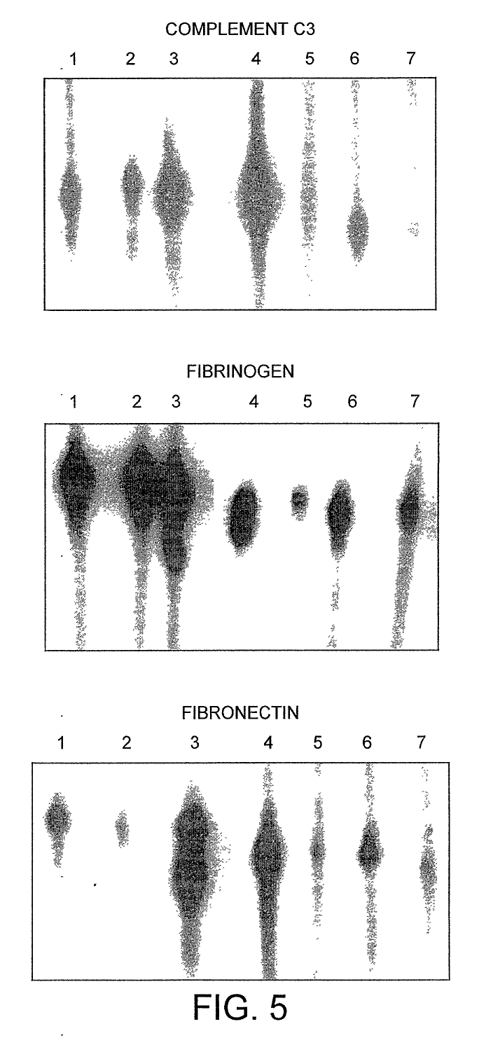

The immunoblot is shown in FIG. 5. It was found that complement C3, fibrinogen and fibronectin bind to the DNA/PEI complexes in the eluate; an effect which is significantly reduced after PEGylation (the complexes were PEGylated as in Example 2) (see tracks 4 and 5). The controls (tracks 6 and 7) served to show the extent to which these proteins bind to the filter membrane when no complex is present. In the plasma probe without DNA complexes the protein is mainly found in the filtrate as expected, while no appreciable amounts of the proteins can be found in the eluate (track 1: human plasma, 3 μl, diluted 1:50; track 2: DNA/PEI+plasma, filtrate, 6 μl; track 3: DNA/PEI+plasma, eluate, 20 μl; track 4: 150 μl plasma, diluted 1:70, filtrate, 6 μl; track 5: 150 μl plasma, diluted 1:70, eluate, 20 μl).

b) Reducing the protein binding to DNA/PEI complexes by modification with M-SPA-PEG

Complexes were mixed together as described in a) and modified with M-SPA-PEG as described in Example 2. The incubation with plasma, filtration, elution and electrophoretic separation were carried out as described in Example 5a. For semiquantitative detection the proteins separated were stained with silver (slightly modified method according to Bloom et al., 1987).

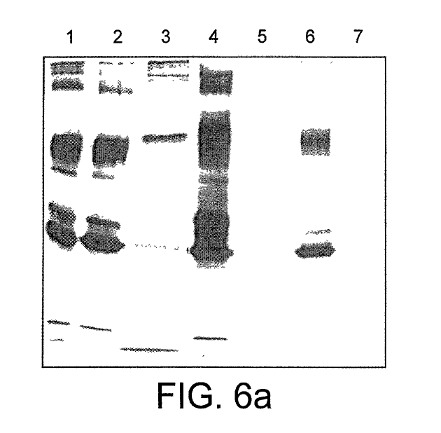

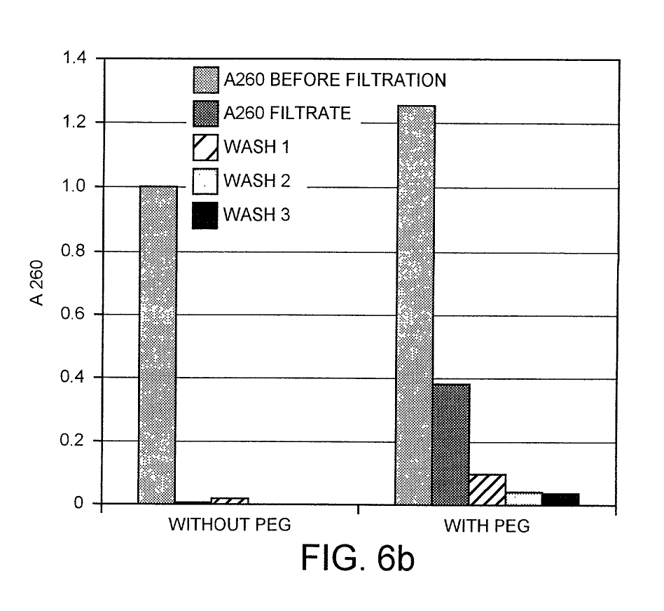

As shown in FIG. 6a, significantly smaller (invisible) amounts of protein bind to PEG-modified complexes (track 5, eluate) than to unmodified complexes (track 3). Track 1: human plasma, 3 μl, diluted 1:50; track 2: DNA/PEI+plasma, filtrate, 6 μl; track 3: DNA/PEI+plasma, eluate, 20 μl; track 4: DNA/PEI-PEG PEG/PEI 9.2/1 (w/w)+plasma, filtrate, 6 μl; track 5: DNA/PEI-PEG PEG/PEI 9.2/1 (w/w)+plasma, eluate, 20 μl; track 6: 150 μl plasma, diluted 1:70, filtrate, 6 μl; track 7: 150 μl plasma, diluted 1:70, eluate, 20 μl.

c) Testing the filterability of DNA/PEI complexes:

In order to ensure that a large amount of the complexes is retained on the membrane after filtration, complexes (DNA concentration of 320 μg/ml) were mixed and PEGylated as described in Example 5a. Then the complexes were filtered through a membrane saturated with BSA and washed 3 times with 300 μl HBS. The absorption of the solution (A260; (absorption peak of nucleic acids) before filtration (A260 before filtration), of the filtrate (A260 filtrate) and of the three washing solutions (wash 1 to wash 3) was measured. FIG. 6b shows that unmodified complexes are completely retained and PEGylated complexes are predominantly retained.

### EXAMPLE 6:

### Effect of PEG modification on gene transfer in mammalian cells

a) Transfection of the human cell line K 562 with PEG-modified DNA/(Tf)PEI complexes

The complexes were mixed as described in Example 1 and modified with M-SPA-PEG as described in Example 2. The DNA concentration during complexing was 20 μg/ml, the ratio of DNA to PEI was N/P 7.2. PEI and Tf-PEI conjugates were used for the DNA complexing, the molar ratio of Tf to PEI in the conjugate was 2/1 (Tf2PEI). The ratio of PEG/PEI was 2.3/1 or 3.7/1 and 7.4/1 (w/w); this corresponds to a molar ratio of 0.25:1, 0.4:1 and 0.8:1, respectively.

The cells (ATCC CCL-243 K-562) were cultivated in RPMI 1640 medium with 100 iU/ml penicillin, 100 μg/ml streptomycin and 10% foetal calf serum (FCS). For each transfection batch, 500,000 cells were seeded in 24-well plates (diameter 22.6 mm, Costar). The transfection was carried out in serum-free medium. After four hours the medium was replaced by serum-containing medium. 24 hours after the start of transfection the cells were removed by centrifuging, harvested in 100 μl of harvesting buffer (250 mM TRIS, pH 7.2, 0.5% Triton X 100), homogenised, centrifuged and 10 μl portions from the supernatant were diluted in 100 μl of probe buffer (25 mM glycylglycine pH 7.8, 5 mM ATP, 15 mM MgCl2) in order to determine the luciferase activity. The measurement was carried out after the injection of 100 μl of injection buffer (200 μM luciferine (Sigma), 20 mM 25 mM glycylglycine pH 7.8) into a Berthold Lumat LB 9507; the results are shown in FIG. 7.

b) Transfection of a murine neuroblastoma cell line with PEG-modified DNA/(Tf)PEI complexes

The complexes were mixed as described in Example 1 and modified with M-SPA-PEG as described in Example 2.

The DNA concentration during complexing was 20 μg/ml, the ratio of DNA to PEI was N/P 7.2. The ratio of PEG/PEI was 3.5/1 or 7.0/1 (w/w); this corresponds to a molar ratio of 0.38:1 or 0.76:1.

PEI and Tf-PEI conjugates were used for the DNA-complexing, the molar ratio of Tf to PEI in the conjugate was 2/1 (Tf₂PEI).

The cells (ATCC CCL 131 Neuro 2A) were cultivated in RPMI 1640 medium with 100 iU/ml penicillin, 100 μg/ml streptomycin and 10% foetal calf serum (FCS). In each transfection batch 300,000 cells were seeded in 6-well plates (diameter 35 mm, Costar). The transfection was carried out in serum-free medium. After four hours the medium was replaced by serum-containing medium. 24 hours after the start of transfection the cells were harvested in 100 μl of harvesting buffer (250 mM TRIS, pH 7.2, 0.5% Triton X 100), homogenised, centrifuged and 10 μl portions were taken from the supernatant and diluted in 100 μl of probe buffer (25 mM glycylglycine pH 7.8, 5 mM ATP,

15 mM MgCl2) in order to determine the luciferase activity. The measurement was carried out after the injection of 100 μl of injection buffer (200 μM luciferine (Sigma), 20 mM 25 mM glycylglycine pH 7.8) into a Berthold Lumat LB 9507.

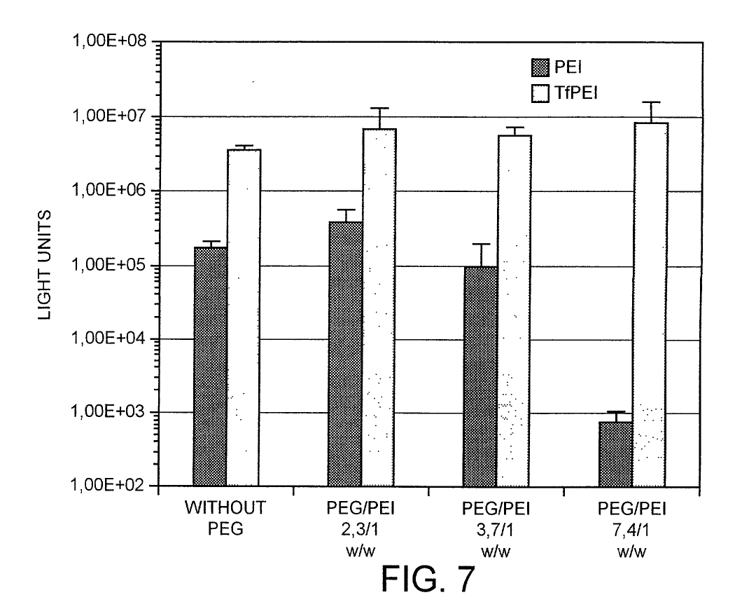

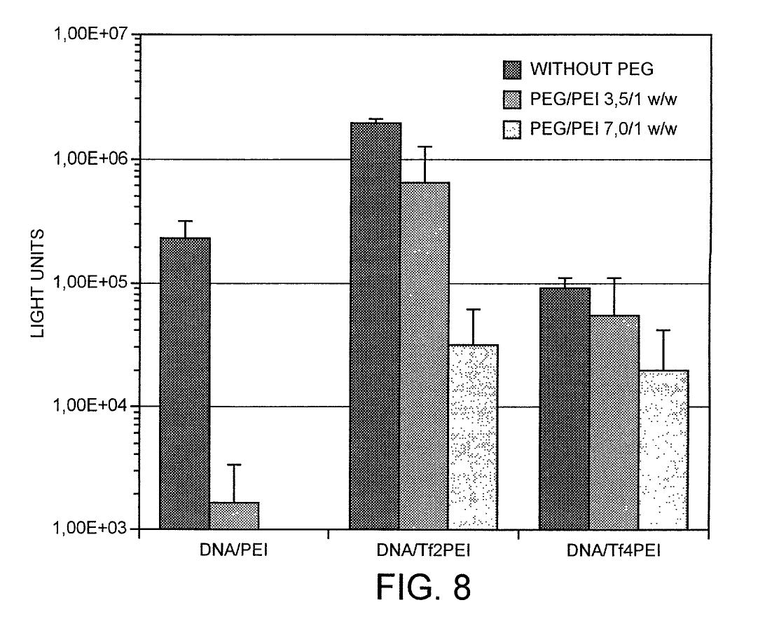

FIGS. 7 and 8 show that modifying DNA/PEI and DNA/TfPEI complexes greatly reduces the non-specific gene transfer (mediated by PEI), whereas receptor-mediated specific gene transfer (mediated by TfPEI) is unaffected (FIG. 7) or affected only slightly, depending on the cell type (FIG. 8).

### EXAMPLE 7:

### Reducing the non-specific uptake of the complexes by P388 murine macrophages by modifying the complexes with PEG

The uptake of the complexes by the cells was carried out with a fluorescence-activated cell sorter (FACS) (FACScan, Becton Dickinson). The excitation wavelength of the laser was 488 nm. The fluorescence was measured at 515 nm.

The DNA concentration during complexing was 320 μg/ml, the N/P value 6.0. The ratio of PEG/PEI was 9.2:1; this corresponds to a molar ratio of 01:1.

The complexes were mixed as described in Example 5a, and modified with M-SPA-PEG, as described in Example 2. Before the complexing the DNA was labelled with YOYO1 (1,1′-(4,4,7,7,-tetramethyl-4,7-diazaundecamethylene)bis-4-[ 3-methyl-2,3-dihydro-(benzo-1,3-oxazole)-2-methylidene ]-quinolinium tetraiodide; Molecular Probes) in a molar ratio of 100:1 (base pairs DNA:YOYO1). The cells were cultivated in DMEM (Dulbeccos modified eagle medium) with 4500 mg/ml glucose, 100 iU/ml penicillin, 100 μg/ml streptomycin and 10% foetal calf serum (FCS). For each batch 300,000 cells were seeded in 35 mm Petri dishes (Falcon No 1008). The incubation with the complexes was carried out in serum-free medium at 37° C. After one hour the cells were washed with PBS and harvested with 5 mM EDTA in PBS.

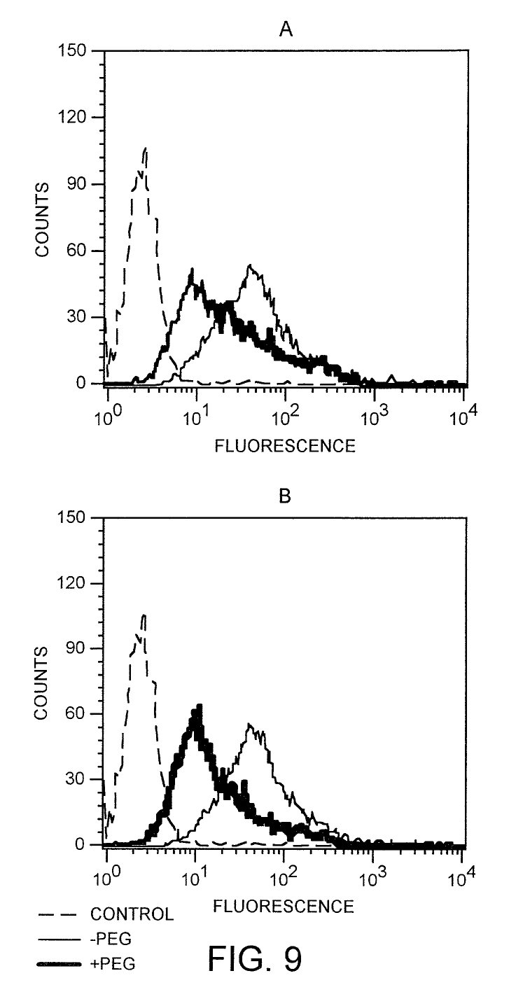

The results of the FACS analysis are shown in FIG. 9 (A: DNA/PEI+/−M-SPA-PEG 37° C., PEG/PEI 9.2/1 w/w). B: DNA/Tf₂PEI+/−M-SPA-PEG 37° C.; PEG/PEI 9.2/1 w/w). The X-axis shows the intensity of fluorescence of the cells measured, the Y-axis the number of events measured. The FACS data show that PEGylation significantly reduces the binding and uptake of the complexes on macrophages. This is demonstrated by the significantly reduced fluorescence of the cells.

### EXAMPLE 8:

### Reducing the interaction with plasma proteins by modifying DNA/Tf-PEI complexes with PEG

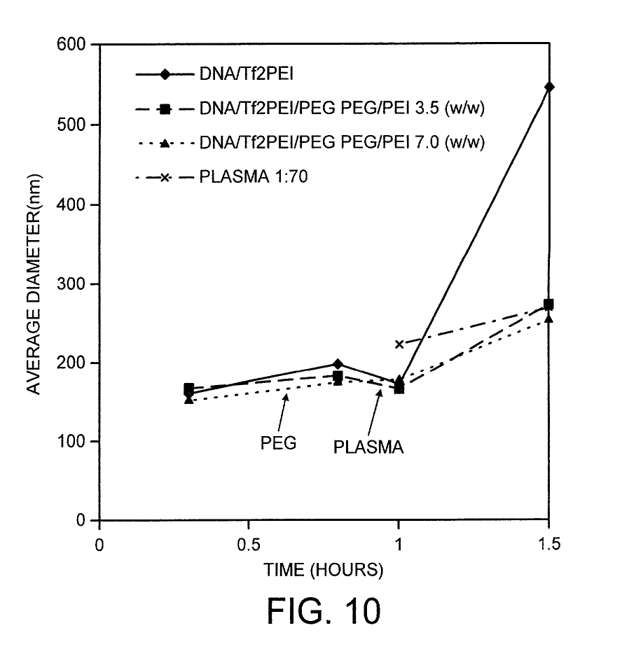

DNA/Tf2-PEI complexes were prepared as described in Example 1 (mixed in water), and modified with PEG as described in Example 2. The DNA concentration was 20 μg/ml, the N/P value was 7.2. The ratio of PEG:PEI was 3.5:1 or 7.0:1 (w/w); this corresponds to a molar ratio of 0.38:1 or 0.76:1. After PEGylation 500 μl of complex were incubated with 7.2 μl plasma at 37° C. At the times specified in FIG. 10 the particle size was measured by LLS. It was found that unmodified complexes form aggregates after incubation with plasma, whereas PEGylated complexes were indistinguishable in size from dilute plasma. Since the tests were carried out in deionised water, the effects of salt could be ruled out.

### EXAMPLE 9

### Preparation of transfection complexes

DNA/TfPEI complexes were prepared and PEGylated as described in Examples 1 and 2. Standard DNA/TfPEI complexes (TfPEI conjugate: molar ratio of about 4 transferrin molecules, bound to PEI, 800 kDa) were mixed with an N/P ratio of 6.0 at a DNA concentration of 100 μg/ml. The complexes were mixed in water or 0.5×HBS (75 mM NaCl, 10 mM HEPES pH 7.4). To ensure iso-osmolarity, glucose was added at a final concentration of 5% or 2.5% (w/v).

PEGylated DNA/TfPEI complexes (DNA/TfPEI/PEG; N/P 6.0, PEG/PEI 10/1 w/w, 1 h PEGylation at room temperature) were mixed at a DNA concentration of 50 μg/ml. The complexes were mixed in water, 0.3×HBS (50 mM NaCl, 7 mM HEPES pH 7.4) or 0.5×HBS. To ensure iso-osmolarity, glucose was added at a final concentration of 5%, 3.3% or 2.5% (w/v). The PEGylated DNA/TfPEI complexes were concentrated, using VIVA-spin-4000-microconcentrators, to a final DNA concentration of 200 μg/ml, as described in Example 4.

### EXAMPLE 10

### PEGylation of DNA/TfPEI complexes increases the stability of the complexes in the blood after use in vivo

a) Use of the transfection complexes in vivo in the animal model

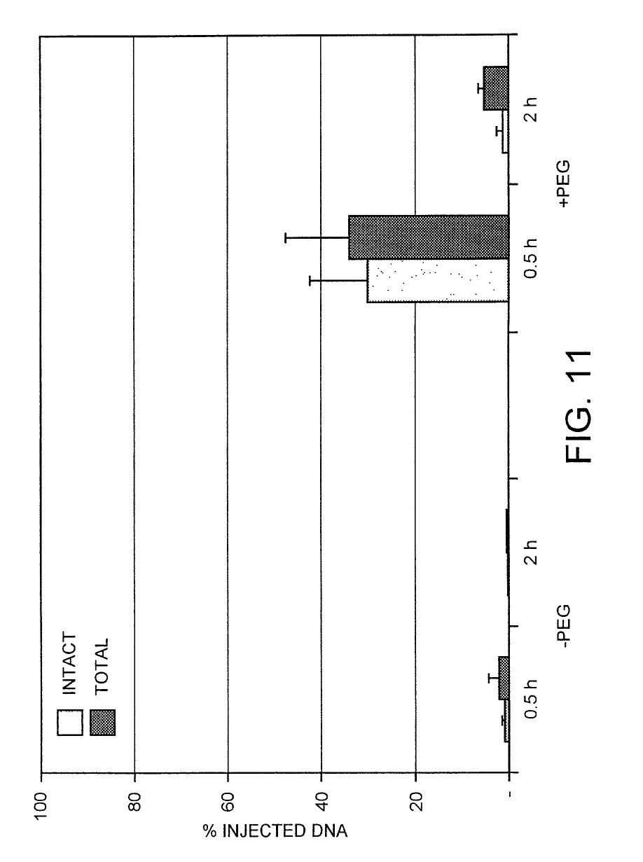

250 μl of PEGylated complexes (containing 50 μg of DNA) or 250 μl of standard complexes (containing 25 μg of DNA) were injected into the caudal vein of female A/J mice (9-12 weeks old). At the times indicated in FIG. 11 after the administration of the transfection complexes the animals were killed by breaking their necks. The blood was collected in Eppendorf test tubes and immediately mixed with sodium citrate in a final concentration of 25 mM. The plasma was separated from the blood cells by centrifugation (10 min, 1000 g at room temperature).

b) Isolation of genomic and plasmid DNA from blood and plasma

The DNA was isolated using the QIAamp Tissue Kit method (Quiagen Cat. No. 29304). 10 μl of heparin (“Novo” heparin, 1000 IU/ml, Novo Nordisk) were added to each aliquot (100 μl) of blood or plasma during the initial incubation at 70° C., in order to ensure the quantitative isolation of plasmid DNA (it had been shown that the complexes dissociate in the presence of heparin).

c) Southern Blotting

The agarose gel was denatured for 45 mins by the standard procedure (Sambrook et al., 1989) (1.5 M NaCl, 0.5 M NaOH), washed with distilled water and rinsed for 30 min in 1 M Tris/1.5 M NaCl. The transfer onto nylon membranes (Gene Screen, DuPont, NEF983) was carried out by capillary transfer in 10×SSC; the DNA was crosslinked by UV radiation onto the filters. The hybridisation and washing were carried out in accordance with the recommendations of the DIG High Prime DNA Labeling and Detection Starter Kit II (Boehringer Mannheim ; Cat. No. 1585614). The filters were prehybridised for 4 h and hybridised overnight with the DIG-labelled probe at 42° C. in 50% formamide, 5×SSC, 0.1% N-lauroylsarcosine, 0.02% SDS, 2% blocking reagent and 100 μg/ml yeast-tRNA. The final wash was carried out in 0.5×SSC, 0.1% SDS at 68° C.

The hybridisation probe was obtained from the plasmid pCMVL (Plank et al., 1992) by DIG labelling according to the manufacturer's instructions (DIG High Prime DNA Labeling and Detection Starter Kit II; Boehringer Mannheim).

The immunological detection was carried out with the substrate in the kit or preferably with Vistra ECF substrate (Amersham Cat. No. RPN5785), which can be quantitatively determined in a Phosphor Imager (Molecular Dynamics). The incubation with the Vistra substrate was carried out overnight.

Estimating the amount of plasmid DNA: different amounts of pCMVL (5 ng, 500 pg, 50 pg, 5 pg or 0.5 pg) were loaded onto each agarose gel in order to compare the intensity of the bands detected on the blots directly. The total quantity of DNA in the plasma was calculated from the values obtained. The results are shown in FIG. 11. This shows that, using standard DNA/TfPEI complexes (without PEGylation), only 1% of the injected DNA (about 300 ng) is detectable in the plasma after 30 minutes. With the PEGylated DNA/TfPEI complexes, more than 20% DNA (10,000 ng) can be detected after a similar time. Two hours after the injection a quantity of DNA which is more than 10 times greater (1500 ng) can be detected with PEGylated complexes than with non-PEGylated standard complexes (100 ng). In both cases some of the DNA is broken down. By using non-PEGylated standard complexes with 50 μg (instead of 25 μg) of DNA, comparable results were obtained (0.5% DNA in the plasma) to those obtained with 25 μg.

### EXAMPLE 11

### Biodistribution of PEGylated DNA/TfPEI complexes after systemic administration

The PEGylated DNA/TfPEI complexes were prepared as described in Example 9; the animal model used was analogous to that in Example 10, but these studies and all the other studies carried out in vivo were performed on tumour-bearing mice. For this purpose, female A/J mice were injected subcutaneously with 2×10⁶ neuroblastoma cells (Neuro2a, ATCC CCL 131). After two weeks, when the tumours had reached a size of about 10 to 14 mm, the transfection complexes were injected into the caudal vein.

a) Administering the transfection complexes in vivo

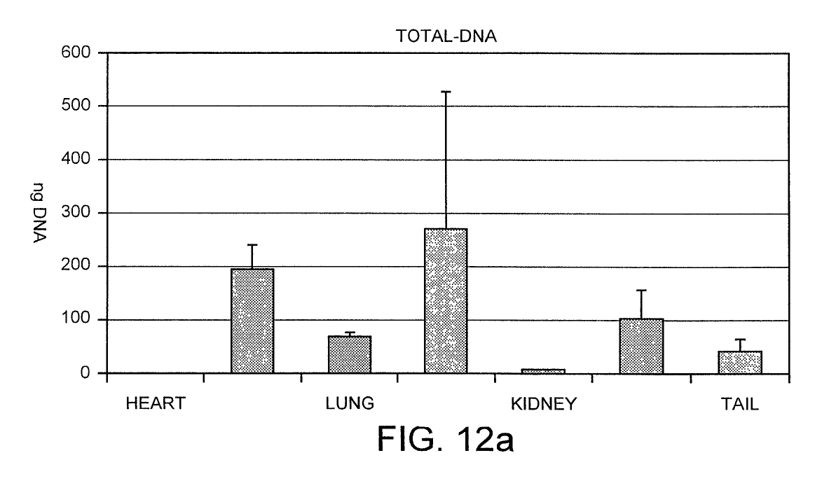

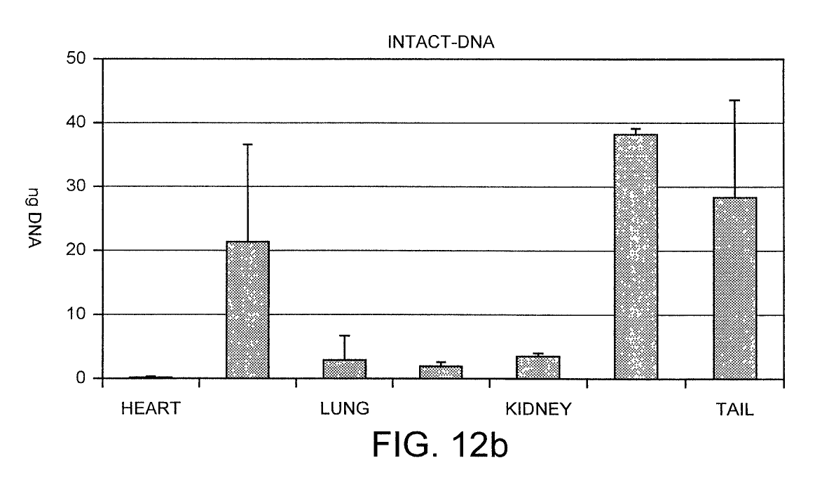

250 μl of PEGylated DNA/TfPEI complexes (containing 50 μg of DNA; N/P=4.8 or 6) were injected into the caudal vein of A/J mice. One day after the administration of the transfection complexes the animals were killed and the tissues specified in FIG. 12 were removed, flash-frozen in liquid nitrogen and stored at −80° C.

b) Isolation of genomic and plasmid DNA

The isolation of the DNA was carried out as described in Example 10 in accordance with the instructions in the QIAamp Tissue Kit. Unlike in Example 10, no heparin was added in this case (the lysing buffer for tissue contained in the kit was sufficient to dissociate the complexes). The precise weight of the mouse organs was determined. 80 μl of PBS/10 mM EDTA were used per 25 mg (spleen: 10 mg) to homogenise the tissues in Dounce homogenisers. 100 μl aliquots (spleen: 250 μl) were used to isolate the DNA.

In order to facilitate the blotting of the total DNA, half the eluted DNA (1/10of the DNA from the mouse tails) was digested with EcoRI (Gibco BRL; 5 h in a total volume of 300 μl with 35 units of EcoRI). The DNA was then precipitated with ethanol, dissolved for some hours in 25 μl of TE (4° C.) and loaded onto a 0.8% agarose gel.

The Southern Blot was carried out as described in Example 10. The total quantity of DNA from each organ was calculated taking into account the total weight of the tissue.

FIG. 12A shows the quantities of pCMVL (intact plus partly degraded) which were detectable in the various tissues by Southern Blot analysis.

FIG. 12B shows the detectable amounts of intact pCMVL. After the systemic administration of PEGylated DNA/TfPEI complexes considerable amounts of DNA were found in the liver, spleen, tail, lungs and in the tumour (small amounts were also found in the kidneys). Interestingly, the largest amounts of intact DNA were found in the tumour, followed by the tail and liver, whereas the majority of the total DNA detected in other organs was degraded (FIG. 12A).

### EXAMPLE 12

### Targeted gene expression in the tumour tissue after systemic administration of PEGylated DNA/TfPEI complexes

The PEGylated DNA/TfPEI complexes were prepared as described in Example 9; the animal model used was identical to that in Example 10.

a) Administration of the transfection complexes in vivo

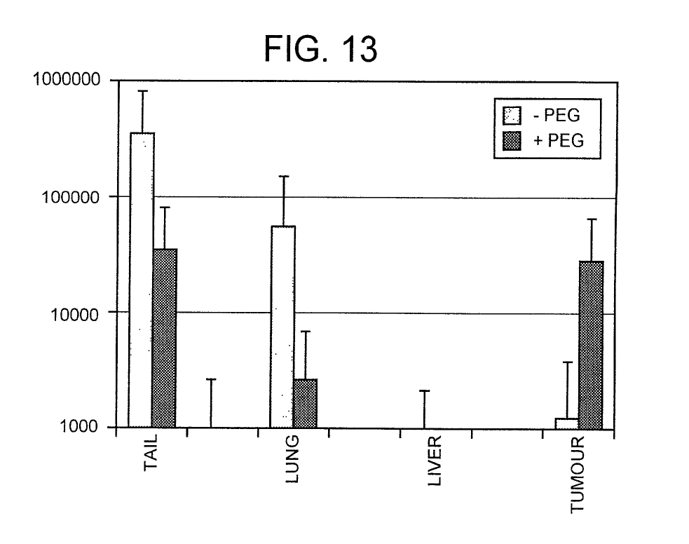

PEGylated DNA/TfPEI complexes (containing 60-80 μg of DNA/200-400 μl; N/P=6; complexes mixed in 0.3×or 0.5×HBS) or non-PEGylated standard DNA/TfPEI complexes (containing 80 μg of DNA/300 μl; N/P=6; complexes mixed in 0.3×or 0.5×HBS) were injected into the caudal vein of A/J mice. Two days after the administration of the transfection complexes the animals were killed and the tissues specified in FIG. 13 were removed. The tissues were homogenised in a buffer containing 250 mM TRIS pH 7.5 using an IKA homogeniser (“Ultraturax”) and flash-frozen in liquid nitrogen. The samples were stored at −80° C. for the luciferase assay.

b) Luciferase assay

The transfection efficiency was determined using a luciferase assay. Samples of homogenised tissue were subjected to three freezing/thawing cycles and centrifuged for 10 min at 10,000 g, in order to pellet the precipitate. The luciferase light units were recorded using a Lumat LB9501/16 (Berthold, Germany) from one aliquot of the supernatant (50 μl) with 10 s integration after automatic injection of the luciferin solution. The luciferase background (300-400 light units) was deducted from each value and the transfection efficiency was expressed as relative light units (Relative Light Units, RLU) per organ/tissue. FIG. 13 shows that, with non-PEGylated standard DNA/TfPEI complexes in the tail and lungs, considerable expression of reporter gene takes place. This could be attributed to the fact that the complexes either remain close to the injection site (tail) or that they aggregate rapidly with plasma proteins and are subsequently filtered out by the lung capillaries. Administering the standard transfection complexes was accompanied by severe acute toxicity. This resulted in approximately 50% mortality in the mice, which could be a consequence of the lung capillaries becoming blocked by the aggregated complexes. Only extremely low gene expression was found in the tumour. In contrast, the systemic administration of the PEGylated DNA/TfPEI complexes resulted in substantial reporter gene expression in the tumour and in the tail. Only minimal expression was detected in the lungs; no expression at all was found in the other organs. The toxicity was significantly reduced compared with the standard complexes.

### EXAMPLE 13

### Measuring the zeta potential: reduced surface loading of PEGylated DNA/TfPEI and DNA/PEI complexes

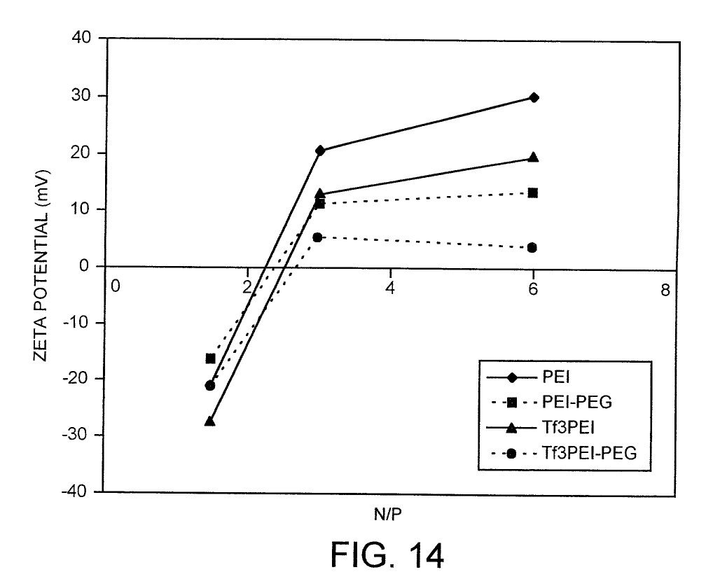

63 μg of DNA in 100 μl of water were complexed with various amounts of TfPEI (N/P 1.5: 12 μg; N/P 3.0: 23 μg; N/P 6.0: 47 μg) in 100 μl. After 30 minutes' complexing the complexes were PEGylated with M-SPA-PEG5000 I (N/P 1.5: 120 μg; N/P 3.0: 230 μg; N/P 6.0: 470 μg. Stock solution 20 mg/ml in DMSO). After 1 hour's PEGylation the complexes were diluted with water (MQ) to a final DNA concentration of 50 μg/ml. The zeta potential was measured in five series of measurements with a ZetaPALS Zeta-Potential-Analyser (Brookhaven) at a field intensity of 13.9 V/cm and 10 Hz using the method described by Miller et al., 1991. The results of the measurements, shown in FIG. 14, show that the incorporation of transferrin in the complex at N/P>3.0 reduces the surface loading. In addition the PEGylation leads to further screening of the surface load from negatively and positively charged complexes.

### EXAMPLE 14:

### Effect of PEG modification on gene transfer in mammalian cells

a) Preparation of small or large transfection complexes

The complexes were mixed as described in Example 1 and modified with M-SPA-PEG as described in Example 2. 10 μg of pCMVL DNA were mixed in 250 μl of buffer with 7.5 μg of PEI (800 kDa) or Tf-PEI conjugate (molar ratio of Tf to PEI in the conjugate 2/1, Tf2PEI) in 250 μl of buffer. The buffer used was either HBG (5% glucose in 10 mM HEPES pH 7.4)—for the small complexes—or HBS (150 mM NaCl, 20 mM HEPES pH 7.4)—for the large complexes. After 40 minutes, 75 μg of M-SPA-PEG5000 were added and the mixture was incubated for another hour at room temperature. Complexes without PEG modification were prepared as controls.

b) Transfection of the human cell line K562 with PEG-modified small or large DNA/(Tf)PEI complexes

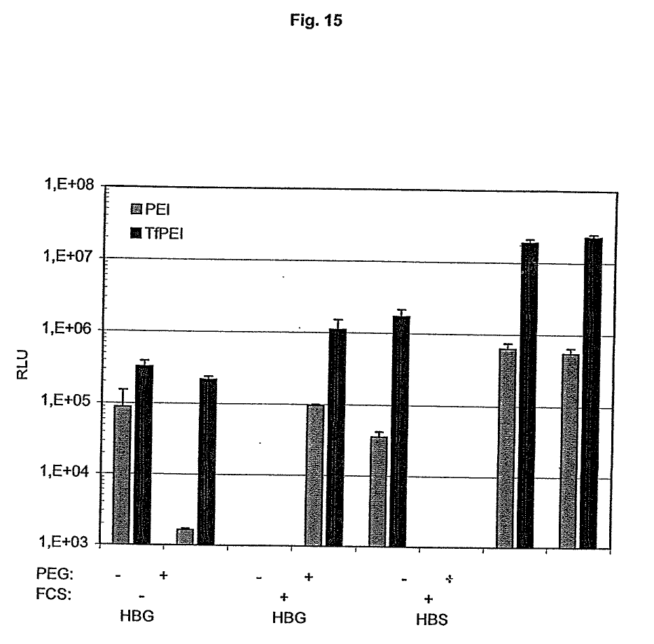

The transfection of the K-562 cells (ATCC CCL-243) was carried out in RPMI 1640 medium with 100 iU/ml penicillin, 100 μg/ml streptomycin and in the presence or absence of 10% foetal calf serum (FCS). For each transfection batch, 500,000 cells were seeded in 24-well plates (diameter 22.6 mm, Costar). The transfection was carried out using 2.5 μg of DNA complex in 125 μl (−FCS batch) or 5 μg of DNA complex in 250 μl (+FCS batch). After four hours the medium was replaced by serum-containing medium. 24 hours after the start of transfection the cells were removed by centrifuging, harvested in 100 μl harvesting buffer and the luciferase expression was determined. The results are shown in FIG. 15 (RLU=Relative light Units). The results show that PEGylation does not have a negative effect on gene transfer efficiency either in small DNA complexes or in large ones, and that in both cases a substantially higher gene transfer is obtained with PEG-transferrin-modified complexes.

### EXAMPLE 15:

### Effect of PEG modification on EGF-mediated gene transfer in mammalian cells

a) Preparation of EGF-PEI conjugates

Conjugates of Epidermal Growth Factor (EGF) with PEI (25 kDa) were prepared by modifying the components with SPDP (Pharmacia 17-0458-01), converting the modified PEI into the mercaptopropionate form and coupling by disulphide bridge formation, analogously to the method described by Kircheis et al, 1997.

4 mg (0.67 μmol) of EGF (EGF1, Serotec, murine) in 1 ml of 16 mM aqueous HEPES buffer (pH 7.9) were left to react with 0.5 ml of a 20 mM ethanolic h at room temperature. This mixture was then dialysed for two days against 50% aqueous ethanol (membrane with molecular weight exclusion limit MWCO 1 kDa, Spectropor 7). The yield of modified EGF was 3.5 mg (87%) in a molar ratio of EGF/pydridinyldithiopropionate of 1:0.8. Analogously, modified EGF was prepared from 1 mg of EGF in a quantity of 0.7 mg.

Mercaptopropionate-modified PEI (10.5 mg, molar ratio of PEI/pydridinyl dithiopropionate of 1:2.8) was obtained by modifying 50 mg of PEI (25 kDa, Aldrich, filtered through Pharmacia Sephadex G25 gel, in 0.76 ml of 0.25 M NaCl, in the form of the hydrochloride, pH 7) with 0.39 ml of a 20 mM ethanolic SPDP solution, after one hour at room temperature followed by gel filtration (Sephadex G25, 10×300 mm column, eluant 0.25 mM NaCl, 20 mM HEPES pH 7.3), reacting some of the intermediate product (20 mg PEI, containing 1.45 μmol of pyridinyl dithiopropionate) with 11 mg of dithiothreitol for one hour under argon and purification by gel filtration (Sephadex G25, 10×100 mm column, eluant 0.25 mM NaCl, 20 mM HEPES pH 7.3, argon-gassed).

Pydridinyl dithiopropionate-modified EGF (4.2 mg EGF, 0.56 μmol pyridinyl dithiopropionate) in 2.2 ml of 50% aqueous ethanol was reacted with mercaptopropionate-modified PEI (7.5 mg PEI, 0.90 μmol of mercapto groups) in 1.1 ml of 0.25 mM NaCl, 20 mM HEPES pH 7.3 under argon. After four days at room temperature the reaction solution was adjusted to 0.5 M NaCl and a total volume of 4 ml by the addition of 3 M NaCl and water and separated by ion exchange chromatography (Biorad Macroprep High S, 100×10 mm, buffer A: 20 mM HEPES pH 7.3; buffer B: 3 M NaCl, 20 mM HEPES pH 7.3; gradient 22% B to 78% B). The product fraction (elution between 2-3 M NaCl) was dialysed against HBS (150 mM NaCl, 20 mM HEPES pH 7.3) and yielded a conjugate of 1.9 mg of EGF modified with 6.35 mg of PEI. This corresponds to a molar ratio EGF/PEI of 1.28:1.

b) Preparation of transfection complexes

The complexes were mixed analogously to the method described in Example 1 and modified with M-SPA-PEG, as described in Example 2. 5 μg of pCMVL DNA were mixed in 125 μl of buffer with 3.75 μg of PEI (25 kDa) as unmodified PEI (hydrochloride), or as a 1:1 (w/w) mixture of unmodified PEI (hydrochloride) with EGF-PEI (cf a)), in 125 μl of buffer. The buffers used were either HBS (150 mM NaCl, 20 mM HEPES pH 7.4) or 0.5×HBS (75 mM NaCl, 10 mM HEPES pH 7.4). After 30 minutes 37.5 μg of M-SPA-PEG5000 were added and the mixture was incubated for a further hour at room temperature. Complexes without PEG modification were prepared as the controls. To ensure iso-osmolarity, glucose was added to the 0.5×HBS complexes in a final concentration of 2.5% (w/v).

c) Transfection of the human cell line KB with PEG-modified DNA/(EGF)PEI complexes

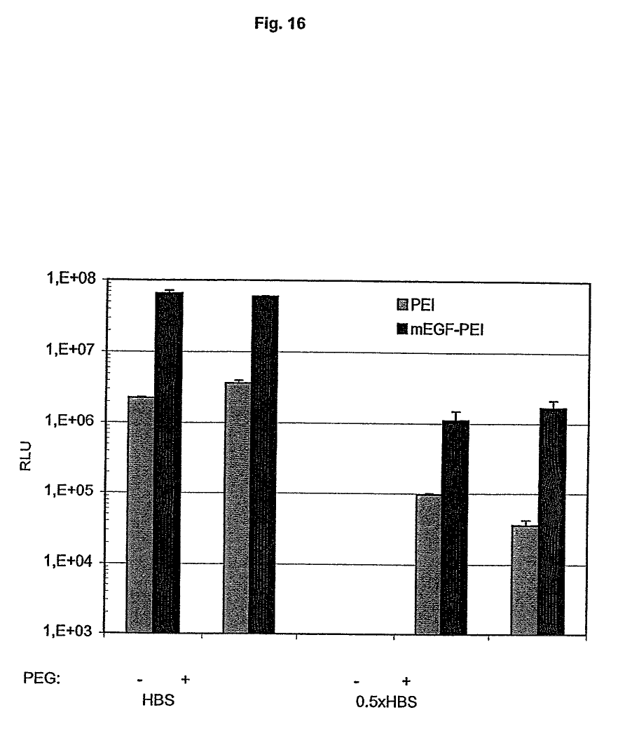

500,000 KB cells (ATCC CCL-17) in T25 flasks (Costar) were seeded for each transfection batch. The transfection was carried out in 2 ml of DMEM medium containing 10% foetal calf serum (FCS) with 5 μg of DNA complex in 250μl solution. After four hours the medium was supplemented with another 2 ml of serum-containing medium. 24 hours after the start of transfection the cells were harvested and the luciferase expression was determined. The results are shown in FIG. 16. The results show that the gene transfer efficiency is maintained even with PEGylation of the DNA complexes prepared in HBS or 0.5×HBS, and that in both cases considerably higher gene transfer is obtained with EGF-modified complexes.

### BIBLIOGRAPHY

Abdallah, B., et al., 1996, Hum Gene Ther 7 (16): 1947-1954

Absolom, D. R., 1986, Methods Enzymol 132; 281-318

Abuchowski et al, 1984, Cancer Biochem. Biophys 7:175

Allen et al, 1991, Biochim Biophys Acta 1066:29

Beauchamp et al, 1983, Anal. Biochem 131:25

Bloom, H., Beier, H., Gross, H. S., 1987, Electrophoresis 8:93-99

Blume et al, 1990, Biochim Biophys. Acta 1029, 91-7

Boussif, O.; Lezoualc'h, F.; Zanta, M. A.; Mergny, M. D.; Scherman, D.; Demeneix, B.; Behr, J. P., 1995, Proc Natl Acad Sci USA 92; 7297-301

Boussif, O, et al., 1996, Gene Ther 3 (12):1074-1080

Chamow et al, 1994, Bioconjugate Chem., 5:133

Chonn, A.; Cullis, P. R.; Devine, D. V., 1991, J Immunol 146; 4234-41

Chonn, A.; Semple, S. C.; Cullis, P. R., 1992, J Biol Chem 267; 18759-65

Delgado et al, 1990, Biotech. Appl. Biochem., 12:119

Dust et al, 1990, Macromolecules, 23:119

Elling et al, 1991, Biotech. Appl. Biochem. 13:354

Harris, J. M., et al., 1989, Polymer Preprints 30 (2): 356

Hodgson, C. P., 1995, Biotechnology 13; 222-5.

Joppich et al, 1979, Macromol. Chem., 180:408

Kircheis, R.; Kichler, A.; Wallner, G.; Kursa, M.; Ogris, M.; Felzmann, T.; Buchberger, M.; Wagner, E., 1997, Gene Therapy 4; 409-18

Kirpotin, et al., 1997, Biochemistry 36, 66-75

Klibanov et al, 1990, FEBS Letters 268:235

Klibanov et al, 1991, Biochem. Biophys. Acta, 1062:142

Mayhew et al, 1992, Int. J. Cancer 51, 1-8

Miller, J., et al., 1991, J Coll Int Sci 143(2)

Moghimi, S. M.; Muir, I. S.; Illum, L.; Davis, S. S.; Kolb Bachofen, V., 1993, Biochim Biophys Acta 1179; 157-65

Mori, A.; Klibanov, A. L.; Torchilin, V. P.; Huang, L., 1991, FEBS Lett 284; 263-6

Nakamura, A., et al., 1986, J Biol. Chem 261:16792

Nilsson et al, 1984, Methods Enzymol., 104: 56

Papahadjopoulos et al, 1991, Proc. Natl. Acad. Sci. USA 88, 11460-4

Pita et al, 1970, Eur. J. Biochem. 94:11

Plank, C., et al., 1992, Bioconjugate Chemistry 3(6): 533-539

Plank, C.; Mechtler, K.; Szoka, F. J.; Wagner, E., 1996, Hum Gene Ther 7:1437-1446

Roitt, I. M.; Brostoff, J.,1991, Male, C. K.:Kurzes Lehrbuch der Immunologie; Thieme Publishing, 2nd Edition

Sarin et al, 1981, Anal. Biochem.117, 147-57

Sambrook, J., et al., 1989, Molecular Cloning: A Laboratory Manual, 2^{nd} Ed., Cold Spring Harbor, N.Y.:Cold Spring Harbor Laboratory Press

Senior, J.; Delgado, C.; Fisher, D.; Tilcock, C.; Gregoriadis, G., 1991, Biochim Biophys Acta 1062; 77-82

Stryer, 1990, Biochemie, Chapter 31, Spectrum of Science Publishing, Heidelberg

Torchilin, V. P.; Klibanov, A. L.; Huang, L.; S, O. D.; Nossiff, N. D.; Khaw, B. A., 1992, Faseb J 6; 2716-9

Torchilin, V. P., et al., 1994, Biochim Biophys Acta 1195, , 181-184

Torchilin, V. P., and Papisov, M. I., 1994, J Liposome Res 4(1), , 725-739

Wirth et al, 1991, Bioorg. Chem., 19: 133

Woodle, M. C.; Newman, M. S.; Cohen, J. A.,1994, J Drug Target 2; 397-403

Yoshinaga et al, 1989, J. Bioactive Comp. Polym., 4:17

Yoshioka, 1991, Biomaterials 12, 861-4

Zalipsky, S. and Barany, G., 1990, J Bioact Compatible Polym 5:227

Zalipsky, S., 1993, Bioconjugate Chemistry 4, 296-299

Zalipsky, S., et al., 1997, Bioconjugate Chemistry 8, 111-118

1. Complexes of nucleic acid and polyethyleneimine (PEI), characterised in that the PEI is modified with a hydrophilic polymer covalently coupled thereto.

2. Complexes according to claim 1, characterised in that the nucleic acid is DNA and the ratio of DNA to PEI, expressed by the molar ratio of the nitrogen atoms in the PEI to the phosphate atoms in the DNA (N/P value), is about 0.5 to about 100.

3. Complexes according to claim 2, characterised in that the N/P value is about 2 to about 20.

4. Complexes according to claim 3, characterised in that the N/P value is about 3 to about 10.

5. Complexes according to one of the preceding claims, characterised in that the PEI has a molecular weight of about 700 D to about 2,000,000 D.

6. Complexes according to claim 5, characterised in that the PEI has a molecular weight of about 2,000 D to about 800,000 D.

7. Complexes according to one of the preceding claims, characterised in that the hydrophilic polymer is linear.

8. Complexes according to one of the preceding claims, characterised in that the hydrophilic polymer is selected from among the group of polyethyleneglycols (PEG), polyvinylpyrollidones, polyacrylamides, polyvinylalcohols, or copolymers thereof.

9. Complexes according to claim 8, characterised in that the hydrophilic polymer is PEG.

10. Complexes according to claim 8 or 9, characterised in that the molecular weight of the hydrophilic polymer is about 500 D to about 20,000 D.

11. Complexes according to claim 10, characterised in that the molecular weight of the hydrophilic polymer is about 1,000 D to about 10,000 D.

12. Complexes according to one of the preceding claims, characterised in that the molar ratio of polymer: primary amino groups/PEI is about 1:10 to about 10:1.

13. Complexes according to claim 12, characterised in that the ratio is about 1:5 to about 5:1.

14. Complexes according to claim 13, characterised in that the ratio is about 1:3 to about 1:1.

15. Complexes according to one of the preceding claims, characterised in that PEI is modified with a cellular ligand.

16. Complexes according to claim 15, characterised in that the ligand is transferrin.

17. Complexes according to claim 15, characterised in that the ligand is EGF.

18. Complexes according to claim 15, characterised in that PEI is bound to the ligand via the hydrophilic polymer.

19. Complexes according to one of the preceding claims, characterised in that they contain, as the nucleic acid, a therapeutically active nucleic acid.

20. Complexes according to claim 19, characterised in that the therapeutically active nucleic acid codes for one or more cytokines.

21. Complexes according to claim 19, characterised in that the therapeutically active nucleic acid codes for one or more tumour antigens or fragments thereof.

22. Complexes according to claim 19, characterised in that the therapeutically active nucleic acid is a suicide gene.

23. Complexes according to claim 22, characterised in that the suicide gene is the Herpes Simplex thymidine kinase gene.

24. Process for preparing complexes according to one of claims 1 to 23, characterised in that first DNA and PEI, optionally modified with a cellular ligand, are complexed by mixing the dilute solutions and then the hydrophilic polymer is bound to PEI.

25. Process according to claim 24, characterised in that the DNA concentration is about 5 to 50 μg of DNA/ml.

26. Process according to claim 25, characterised in that the DNA concentration is about 10 to 40 μg of DNA/ml.

27. Process according to claim 25 or 26, characterised in that the complexing is carried out at a salt concentration below the physiological value.

28. Process according to claim 27, characterised in that the complexing is carried out in deionised water.

29. Preparation process according to one of claims 24 to 28, characterised in that after the complexing of DNA and optionally modified PEI, the complexes of the dilute solution are adjusted to a concentration of about 200 μg/ml to 1 mg/ml, based on DNA.

30. Composition for the transfection of mammalian cells, characterised in that it contains one or more complexes according to one of claims 1 to 23 in a concentration of 200 μg/ml to 1 mg/ml, based on DNA.

31. Pharmaceutical composition containing one or more complexes according to claim 19.

32. Pharmaceutical composition according to claim 31, characterised in that it contains the complexes in a concentration of about 200 μg/ml to about 1 mg/ml, based on DNA.

33. Pharmaceutical composition according to claim 31 or 32, characterised in that the complexes contain DNA which codes for one or more cytokines.

34. Pharmaceutical composition according to claim 31 or 32 in the form of a tumour vaccine, characterised in that the complexes contain DNA which codes for one or more tumour antigens or fragments thereof, optionally combined with DNA which codes for one or more cytokines.
